(19) United States
(12) Patent Application Publication
(10) Pub. No.: US 2023/0137591 A1
(43) Pub. Date: May 4, 2023

(54) ENDOSCOPE TREATMENT TOOL, AND TREATMENT METHOD

(71) Applicant: OLYMPUS MEDICAL SYSTEMS CORP., Tokyo (JP)

(72) Inventors: Kotaro YAMADA, Tokyo (JP); Yusuke SHIOTA, Tokyo (JP); Hiromasa KATO, Tokyo (JP)

(73) Assignee: OLYMPUS MEDICAL SYSTEMS CORP., Tokyo (JP)

(21) Appl. No.: 17/973,825

(22) Filed: Oct. 26, 2022

Related U.S. Application Data
(60) Provisional application No. 63/273,172, filed on Oct. 29, 2021.

(51) Int. Cl. A61B 18/14; A61B 17/00

(52) U.S. Cl. CPC A61B 18/1477; A61B 17/00234; A61B 2017/00269; A61B 2018/1475; A61B 2018/00601

(57) ABSTRACT

An endoscope treatment tool includes a sheath, an electrode and a needle. The sheath includes a first hole opened at a distal end of the sheath. The electrode is inserted into the first hole and includes a second hole opened at a distal end of the electrode. The needle is inserted into the second hole. The needle is movable to protrude from a distal end of the second hole of the electrode. A gap between the electrode and the needle is configured to pass a fluid.

### RELATED APPLICATION DATA

This application is based on and claims priority under 35 U.S.C. § 119 to U.S. Provisional Application No. 63/273,172, filed Oct. 29, 2021, the entire contents of which are incorporated herein by reference.

### FIELD OF THE DISCLOSURE

The disclosure relates to an endoscope treatment tool, and a treatment method.

### BACKGROUND

In Endoscopic Submucosal Dissection (ESD), treatment tools for incision and dissection, such as high frequency knives, and treatment tools for local injection and hemostasis are used (see, for example, CN 111202485 A, JP 2012-523863 A, and CN 108272503 A).

CN 111202485 A and JP 2012-523863 A disclose treatment tools for an endoscope, which are capable of performing tissue incision treatment and local injection treatment.

In addition, in a case where local injection treatment is performed, as disclosed in CN 108272503 A, a method of locally injecting a liquid into a tissue by discharging the liquid from a distal end of an electrode for incision or dissection is known.

### SUMMARY

In some embodiments, an endoscope treatment tool includes a sheath, an electrode and a needle. The sheath includes a first hole opened at a distal end of the sheath. The electrode is inserted into the first hole and includes a second hole opened at a distal end of the electrode. The needle is inserted into the second hole. The needle is movable to protrude from a distal end of the second hole of the electrode. A gap between the electrode and the needle is configured to pass a fluid.

### BRIEF DESCRIPTION OF THE DRAWINGS

FIG. 1 is a diagram illustrating an endoscope system according to a first embodiment.

FIG. 2 is a diagram for describing a configuration of a treatment tool insertion portion.

FIG. 3 is a diagram for describing a configuration of a treatment tool insertion portion.

FIG. 4 is a diagram for describing a configuration of a treatment tool insertion portion.

FIG. 5A is a diagram illustrating an example of a shape of a protrusion portion.

FIG. 5B is a diagram illustrating an example of a shape of a protrusion portion.

FIG. 5C is a diagram illustrating an example of a shape of a protrusion portion.

FIG. 6A is a diagram illustrating an example of a shape of a flange portion.

FIG. 6B is a diagram illustrating an example of a shape of a flange portion.

FIG. 6C is a diagram illustrating an example of a shape of a flange portion.

FIG. 6D is a diagram illustrating an example of a shape of a flange portion.

FIG. 7 is a diagram for describing an operation of an endoscope treatment tool.

FIG. 8 is a diagram for describing an operation of an endoscope treatment tool.

FIG. 9 is a diagram for describing an operation of an endoscope treatment tool.

FIG. 10 is a diagram for describing an operation of an endoscope treatment tool.

FIG. 11 is a diagram illustrating an example of a configuration in a case where a tube is not provided.

FIG. 12 is a diagram illustrating a modification of the first embodiment.

FIG. 13 is a diagram illustrating a modification of the first embodiment.

FIG. 14 is a diagram for describing a configuration of a treatment tool insertion portion according to a second embodiment.

FIG. 15 is a diagram for describing a configuration of a treatment tool insertion portion according to the second embodiment.

FIG. 16 is a diagram for describing a configuration of a treatment tool insertion portion according to the second embodiment.

FIG. 17 is a diagram for describing a configuration of a treatment tool insertion portion according to a third embodiment.

FIG. 18 is a diagram for describing a configuration of a treatment tool insertion portion according to the third embodiment.

FIG. 19 is a diagram for describing a configuration of a treatment tool insertion portion according to the third embodiment.

FIG. 20 is a diagram for describing a configuration of a treatment tool insertion portion according to a fourth embodiment.

FIG. 21 is a diagram for describing a configuration of a treatment tool insertion portion according to the fourth embodiment.

FIG. 22 is a diagram for describing a configuration of a treatment tool insertion portion according to the fourth embodiment.

FIG. 23 is a diagram illustrating a first modification of the first to fourth embodiments.

FIG. 24 is a diagram illustrating a second modification of the first to fourth embodiments.

FIG. 25 is a diagram illustrating a third modification of the first to fourth embodiments.

FIG. 26 is a diagram illustrating a fourth modification of the first to fourth embodiments.

FIG. 27 is a diagram illustrating a fifth modification of the first to fourth embodiments.

### DETAILED DESCRIPTION

Hereinafter, modes for carrying out the disclosure (hereinafter referred to as embodiments) will be described with reference to the drawings. It is noted that the disclosure is not limited to the embodiments described below. Furthermore, in the description of the drawings, the same reference numerals are assigned to the same parts.

### First Embodiment

Configuration of Endoscope System

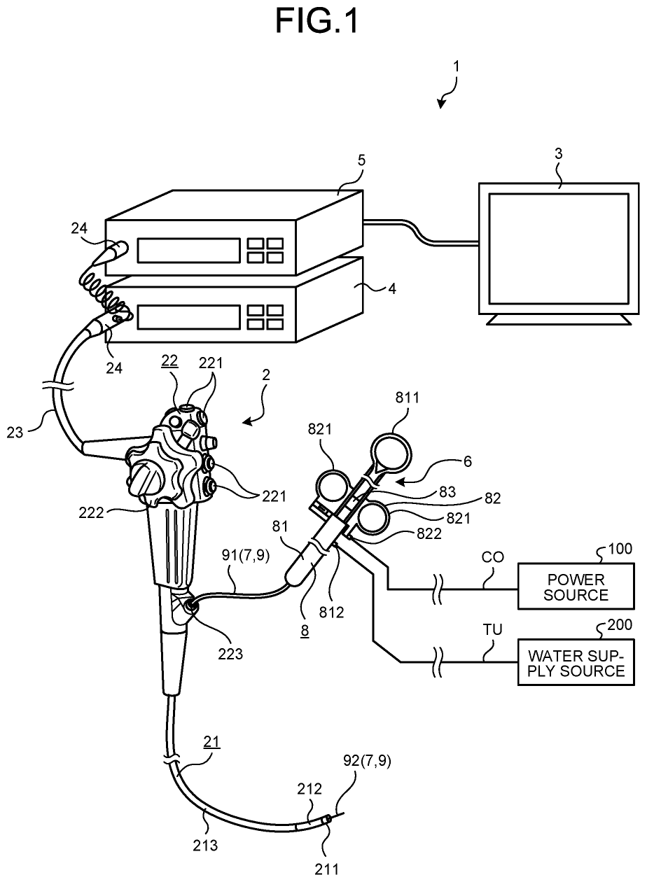

FIG. 1 is a diagram illustrating an endoscope system 1 according to a first embodiment.

The endoscope system 1 is a system that is used in the medical field and treats a site to be treated in a living tissue inside a body cavity (hereinafter referred to as a target site) by applying high frequency energy to the target site while observing the body cavity. It is noted that the treatment that can be performed by the endoscope system 1 according to the first embodiment is a treatment such as coagulation (sealing) of the target site or incision of the target site. As illustrated in FIG. 1, the endoscope system 1 includes an endoscope 2, a display device 3, a light source device 4, a control device 5, and a treatment tool 6 for an endoscope.

The endoscope 2 is partially inserted into the body cavity, captures a subject image reflected from the body cavity, and outputs an image signal generated by the image capture. As illustrated in FIG. 1, the endoscope 2 includes an endoscope insertion portion 21, an endoscope operating portion 22, a universal cord 23, and a connector portion 24.

The endoscope insertion portion 21 is a portion that is at least partially flexible and is inserted into the body cavity. As illustrated in FIG. 1, the endoscope insertion portion 21 includes a distal end unit 211, a bendable portion 212, and a flexible tube 213.

The distal end unit 211 is provided at the distal end of the endoscope insertion portion 21. Although the specific illustration is omitted, the distal end unit 211 is provided with an illumination optical system, an imaging optical system, and an imaging unit.

The illumination optical system faces one end of a light guide (not illustrated) routed in the endoscope insertion portion 21 and irradiates light transmitted by the light guide onto the inside of the body cavity from the distal end of the endoscope insertion portion 21.

The imaging optical system irradiates light onto the inside of the body cavity from the illumination optical system, takes in the light (subject image) reflected from the inside of the body cavity, and forms an image on an imaging surface of an image sensor constituting the imaging unit.

The imaging unit is configured to include an image sensor, such as a Charge Coupled Device (CCD) or a Complementary Metal Oxide Semiconductor (CMOS). The imaging unit captures a subject image formed by the imaging optical system and outputs an image signal generated by the image capture.

The bendable portion 212 is connected to the proximal end side (endoscope operating portion 22 side) of the distal end unit 211. Although the specific illustration is omitted, the bendable portion 212 has a configuration in which a plurality of curved pieces are connected to each other and the bendable portion 212 can be curved.

The flexible tube 213 is connected to the proximal end side (endoscope operating portion 22 side) of the bendable portion 212 and has an elongated shape and flexibility.

The endoscope operating portion 22 is connected to the proximal end portion of the endoscope insertion portion 21. The endoscope operating portion 22 accepts various operations on the endoscope 2. As illustrated in FIG. 1, the endoscope operating portion 22 is provided with a plurality of operation members 221, a curved knob 222, and an insertion port 223.

The plurality of operation members 221 are configured by buttons or the like that accept various operations.

The curved knob 222 is configured to be rotatable according to a user operation. Therefore, the curved knob 222 rotates to operate a bending mechanism (not illustrated) of a metal or resin wire or the like disposed in the endoscope insertion portion 21. Due to this, the bendable portion 212 is curved.

The insertion port 223 is an insertion port which communicates with a pipeline (not illustrated) extending from the distal end to the proximal end side (endoscope operating portion 22 side) of the endoscope insertion portion 21 and allows a treatment tool insertion portion 7 or the like in the treatment tool 6 for an endoscope to be inserted into the pipeline from the outside.

The universal cord 23 is a cord which extends from the endoscope operating portion 22 in a direction different from the extension direction of the endoscope insertion portion 21 and in which the above-described light guide, a signal line for transmitting the above-described image signal, and the like are disposed.

The connector portion 24 is provided at the end of the universal cord 23 and is detachably connected to the light source device 4 and the control device 5.

The display device 3 is a Liquid Crystal Display (LCD), an Electro Luminescence (EL) display, or the like, and displays a predetermined image under the control of the control device 5.

The light source device 4 emits illumination light. The illumination light, which is emitted from the light source device 4, passes through the connector portion 24, the universal cord 23, the endoscope operating portion 22, and the light guide and the illumination optical system routed in the endoscope insertion portion 21, and is then irradiated from the distal end of the endoscope insertion portion 21 toward the inside of the body cavity.

The control device 5 is configured to include a Central Processing Unit (CPU), a Field-Programmable Gate Array (FPGA), and the like, and controls overall operations of the display device 3 and the light source device 4.

For example, the control device 5 generates an endoscopic image by performing predetermined processing on the image signal input from the above-described imaging unit through the above-described signal line. The control device 5 controls the operation of the display device 3 and causes the display device 3 to display the endoscopic image or the like.

It is noted that, in the first embodiment, the light source device 4 and the control device 5 are configured separately, but may be integrally provided in one housing.

Configuration of treatment tool for endoscope The treatment tool 6 for an endoscope is, for example, a treatment tool used in Endoscopic Submucosal Dissection (ESD). As illustrated in FIG. 1, the treatment tool 6 for an endoscope includes the treatment tool insertion portion 7 and a treatment tool operating portion 8.

As illustrated in FIG. 1, the treatment tool insertion portion 7 is a portion that passes through the pipeline in the endoscope insertion portion 21 from the insertion port 223, protrudes from the distal end of the endoscope insertion portion 21, and is inserted into the body cavity. The treatment tool insertion portion 7 corresponds to an insertion portion.

It is noted that the detailed configuration of the treatment tool insertion portion 7 will be described in “Configuration of treatment tool insertion portion” described below. In addition, the “distal end” shown below means one end of the treatment tool insertion portion 7 in the insertion direction, and the “proximal end” shown below means the other end of the treatment tool insertion portion 7 on the side opposite to the insertion direction.

The treatment tool operating portion 8 is connected to the proximal end portion of the treatment tool insertion portion 7. The treatment tool operating portion 8 receives the operation on the treatment tool 6 for an endoscope. As illustrated in FIG. 1, the treatment tool operating portion 8 includes an operating portion body 81, a first slider 82, and a second slider 83.

The operating portion body 81 has an elongated shape, and a proximal end portion of a sheath 9, described below, constituting the treatment tool insertion portion 7 is fixed thereto. In addition, as illustrated in FIG. 1, a ring 811 for an operator such as an operating surgeon to hang his or her finger is provided at the proximal end portion of the operating portion body 81. Furthermore, the operating portion body 81 is provided with a water supply port 812 to which a tube TU is connected. A physiological saline solution is supplied from a water supply source 200, such as a pump, to the water supply port 812 via the tube TU.

Here, the physiological saline solution corresponds to a fluid. It is noted that the fluid is not limited to the physiological saline solution, and other liquids and gases such as air may be adopted.

The first slider 82 is attached to the operating portion body 81 so as to be movable along the longitudinal direction of the operating portion body 81 according to an operation by an operator such as an operating surgeon. As illustrated in FIG. 1, the first slider 82 is provided with a pair of rings 821 for an operator such as an operating surgeon to hang his or her finger. In addition, the first slider 82 is provided with a plug 822 to which a power cord CO is connected. The plug 822 is electrically connected to a power source 100 via the power cord CO.

The second slider 83 is attached to the operating portion body 81 so as to be movable along the longitudinal direction of the operating portion body 81 according to an operation by an operator such as an operating surgeon. It is noted that the second slider 83 is movable along the longitudinal direction of the operating portion body 81 in a state of being independent of the first slider 82.

Configuration of Treatment Tool Insertion Portion

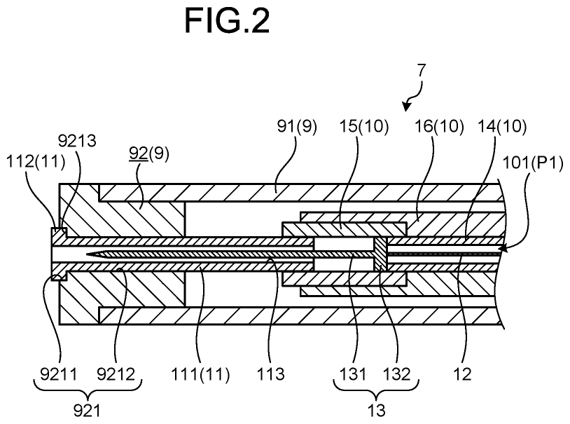

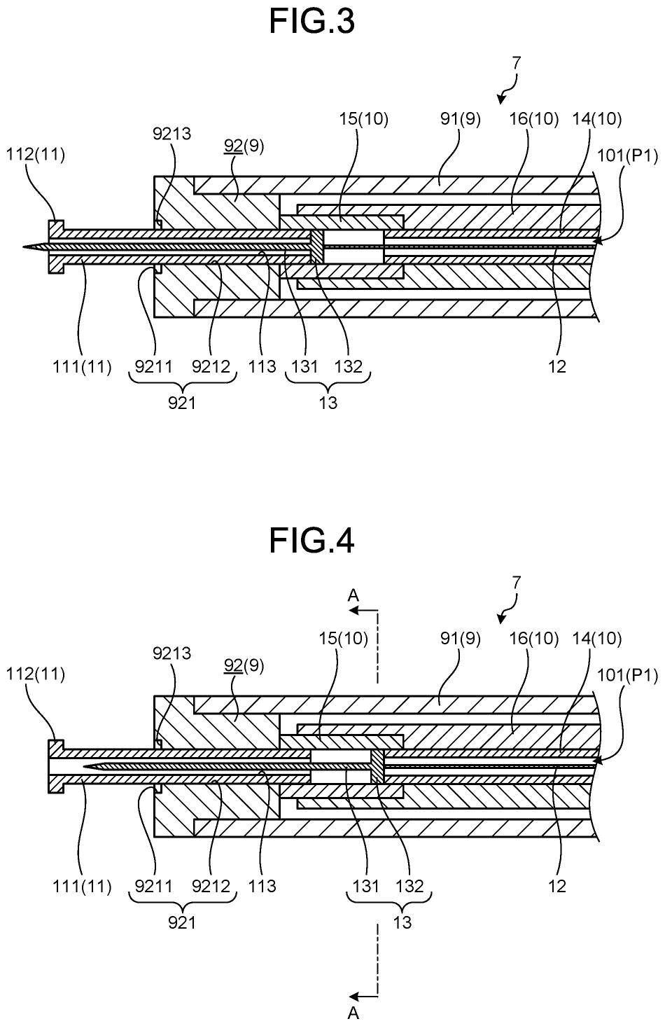

FIGS. 2 to 4 are diagrams for describing the configuration of the treatment tool insertion portion 7. Specifically, FIGS. 2 to 4 are cross-sectional views in which the distal end portion of the treatment tool insertion portion 7 is cut by a plane including the central axis of the treatment tool insertion portion 7. In addition, FIG. 2 is a diagram illustrating the treatment tool insertion portion 7 set to a first state. FIG. 3 is a diagram illustrating the treatment tool insertion portion 7 set to a second state. FIG. 4 is a diagram illustrating the treatment tool insertion portion 7 set to a third state.

As illustrated in FIGS. 1 to 4, the treatment tool insertion portion 7 includes a sheath 9, a first advancing/retreating portion 10 (FIGS. 2 to 4), a knife 11 (FIGS. 2 to 4), a second advancing/retreating portion (wire) 12 (FIGS. 2 to 4), and a needle portion 13 (FIGS. 2 to 4).

The sheath 9 is a portion that constitutes the outer surface of the treatment tool insertion portion 7. As illustrated in FIGS. 1 to 4, the sheath 9 includes a sheath body 91 and a distal end part 92.

The sheath body 91 is a cylindrical member made of a resin material or the like and having insulating properties and flexibility. The proximal end portion of the sheath body 91 is fixed to the operating portion body 81.

The distal end part 92 is configured by a substantially cylindrical member. The distal end part 92 may be made of a member having electrical insulating properties, including ceramic, a resin material, rubber, or the like, or may be made of a member on which insulating coating or the like is performed on a surface of a metal or the like. The distal end part 92 is fixed to the distal end portion of the sheath body 91. More specifically, the distal end part 92 is inserted through the distal end portion of the sheath body 91 and closes the distal end portion thereof. As illustrated in FIGS. 2 to 4, the distal end part 92 is provided with a first hole 921 that communicates with the inside and outside of the sheath 9.

The first hole 921 has a circular cross-sectional shape, is located on the central axis of the distal end part 92, and extends linearly along the central axis thereof. In addition, the first hole 921 has a stepped shape in which the distal end portion has a larger inner diameter than the proximal end portion. In the following, for convenience of explanation, a portion of the first hole 921 having a large inner diameter on the distal end side is referred to as a large diameter portion 9211 (FIGS. 2 to 4), and a portion of the first hole 921 having a small inner diameter on the proximal end side is referred to as a small diameter portion 9212 (FIGS. 2 to 4). In addition, a stepped portion between the large diameter portion 9211 and the small diameter portion 9212 is referred to as a first stepped portion 9213 (FIGS. 2 to 4).

The first advancing/retreating portion 10 is a member that has a distal end portion connected to the knife 11, is capable of advancing and retreating in the sheath body 91, and advances and retreats the knife 11 along the central axis of the sheath 9. As illustrated in FIGS. 2 to 4, the first advancing/retreating portion 10 includes a tube 14, a connecting portion 15, and an inner tube 16. That is, the first advancing/retreating portion 10 can be said as “a tube” as a whole.

The tube 14 is a cylindrical flexible coil that is made of, for example, a conductive material such as a metal, is located inside the sheath 9, and extends along the central axis of the sheath 9. The proximal end portion of the tube 14 is fixed to the first slider 82. That is, the first advancing/retreating portion 10 advances and retreats in the sheath body 91 according to an operation on the first slider 82 by an operator such as an operating surgeon. In addition, the tube 14 is electrically connected to the plug 822. Furthermore, the inside of the tube 14 communicates with the water supply port 812.

It is noted that the metal tube 14 is not always essential if there is a means for electrically conducting the plug 822 and the knife 11 with a metal wire or the like.

The connecting portion 15 is a member that connects the first advancing/retreating portion 10 to the knife 11. The connecting portion 15 is made of a conductive material such as a metal, is located inside the sheath 9, and has a cylindrical shape extending linearly along the central axis thereof. As illustrated in FIGS. 2 to 4, the tube 14 is fixed to the proximal end side of the connecting portion 15 in a state of being inserted therethrough. Due to this, the inside of the tube 14 communicates with the inside of the connecting portion 15. That is, the connecting portion 15 advances and retreats in the sheath body 91 together with the tube 14 according to an operation on the first slider 82 by an operator such as an operating surgeon. On the other hand, the knife 11 is fixed to the distal end side of the connecting portion 15 in a state of being inserted therethrough. That is, the connecting portion 15 is fixed on the outer peripheral surface of the proximal end portion of the knife 11, the knife 11 becomes the whole body together with the tube 14 and the connecting portion 15 according to the operation of the first slider 82, and the distal end of the connecting portion 15 is configured to abut on the distal end part 92 so that the amount of protrusion of the knife 11 from the distal end part 92 is restricted.

The inside of the tube 14 and the inside of the connecting portion 15 described above correspond to a communication hole 101 (FIGS. 2 to 4). The communication hole 101 functions as a passage P1 (FIGS. 2 to 4), through which the physiological saline solution supplied from the water supply source 200 flows via the tube TU and the water supply port 812. That is, the first advancing/retreating portion 10 includes an internal channel that forms a passage communicating with a gap between the knife 11 and the needle portion 13.

The inner tube 16 is a cylindrical member made of a resin material or the like and having insulating properties and flexibility and is provided so as to cover the outer peripheral surface of the tube 14 and the outer peripheral surface of the connecting portion 15. The inner tube 16 prevents the physiological saline solution from leaking out from the passage P1. That is, the inner tube 16 advances and retreats in the sheath body 91 together with the tube 14 and the connecting portion 15 according to an operation on the first slider 82 by an operator such as an operating surgeon.

The knife 11 may correspond to an electrode. The knife 11 injects the physiological saline solution into the target site via a cut made by the needle portion 13 with respect to the target site. The needle portion 13 may correspond to a needle. In addition, the knife 11 is made of a conductive material such as a metal, and the proximal end portion is fixed in a state of being inserted into the connecting portion 15. That is, the knife 11 advances and retreats in the sheath body 91 together with the first advancing/retreating portion 10 according to an operation on the first slider 82 by an operator such as an operating surgeon. In addition, the knife 11 protrudes from the first hole 921 to the outside of the distal end part 92. The knife 11 is energized with a high frequency current from the power source 100 via the power cord CO, the plug 822, the tube 14, and the connecting portion 15, and incises the target site in the body cavity. As illustrated in FIGS. 2 to 4, the knife 11 includes a knife body 111 and a protrusion portion 112.

The knife body 111 is located on the central axis of the distal end part 92 and is configured by a cylindrical member extending along the central axis thereof. Here, as illustrated in FIGS. 2 to 4, the outer diameter dimension of the knife body 111 is set to be slightly smaller than the inner diameter dimension of the first hole 921.

The protrusion portion 112 is provided at the distal end of the knife body 111 and has a disk shape that is coaxial with the central axis of the knife body 111. The maximum width dimension of the protrusion portion 112 is set to be larger than the outer diameter dimension of the knife body 111 and slightly smaller than the inner diameter dimension of the large diameter portion 9211. It is noted that the protrusion portion 112 protrudes and extends radially from the outer peripheral surface of the knife body 111.

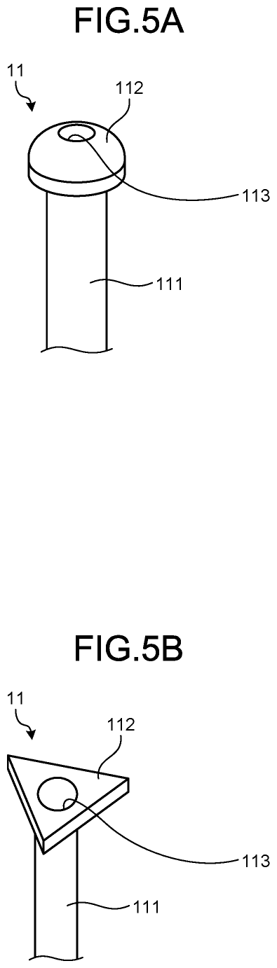

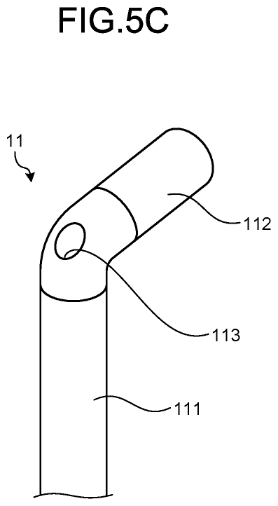

FIGS. 5A to 5C are diagrams illustrating an example of the shape of the protrusion portion 112.

Here, the protrusion portion 112 does not necessarily have to have a disk shape. As illustrated in FIGS. 5A to 5C, the protrusion portion 112 may have a flange shape such as a hemispherical shape (FIG. 5A) or a triangular shape (FIG. 5B). The protrusion portion 112 is not limited to the flange shape, and may have a hook shape (FIG. 5C).

As illustrated in FIGS. 2 to 4, the knife 11 described above is provided with a second hole 113 that is located on the central axis of the knife body 111 and penetrates from the proximal end to the distal end of the knife 11 along the central axis thereof. The second hole 113 communicates with the passage P1 by connecting the knife 11 to the first advancing/retreating portion 10.

The second advancing/retreating portion 12 is, for example, a wire made of a metal material, and is a member that is connected to the proximal end portion of the knife 11, can advance and retreat relative to the first advancing/retreating portion 10 in the communication hole 101, and advances and retreats the needle portion 13 along the central axis of the sheath 9. The second advancing/retreating portion 12 is fixed to the second slider 83. That is, the second advancing/retreating portion 12 advances and retreats in the communication hole 101 according to an operation on the second slider 83 by an operator such as an operating surgeon. The second advancing/retreating portion 12 may correspond to a transmission member.

The needle portion 13 is a solid body, is used for making a cut in the target site (tissue), and is capable of advancing and retreating in the second hole 113. As illustrated in FIGS. 2 to 4, the needle portion 13 includes a needle portion body 131 and a flange portion 132. The needle portion body 131 may correspond to a needle body. The flange portion 132 may correspond to a flange.

The needle portion body 131 is located inside the second hole 113 and is configured by a cylindrical member extending linearly along the central axis of the second hole 113. In addition, the distal end portion of the needle portion body 131 has an outer diameter dimension that becomes smaller toward the distal end, and constitutes a cutting edge. It is noted that the needle portion 13 is provided so as to be able to advance and retreat in a direction orthogonal to the extension direction of the protrusion portion 112.

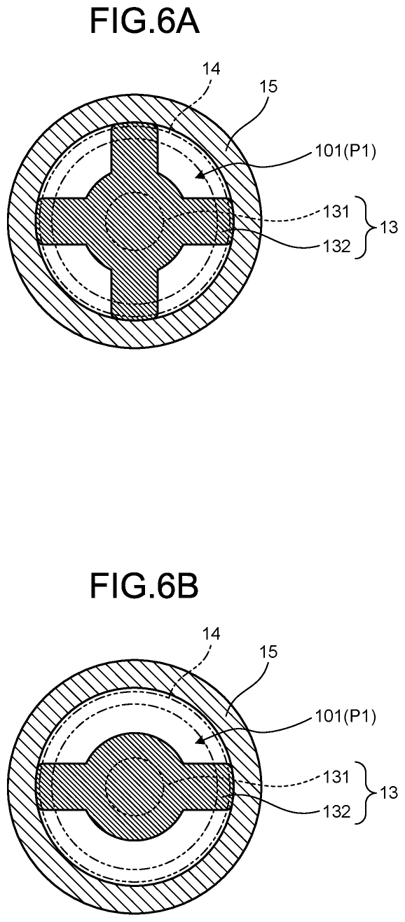

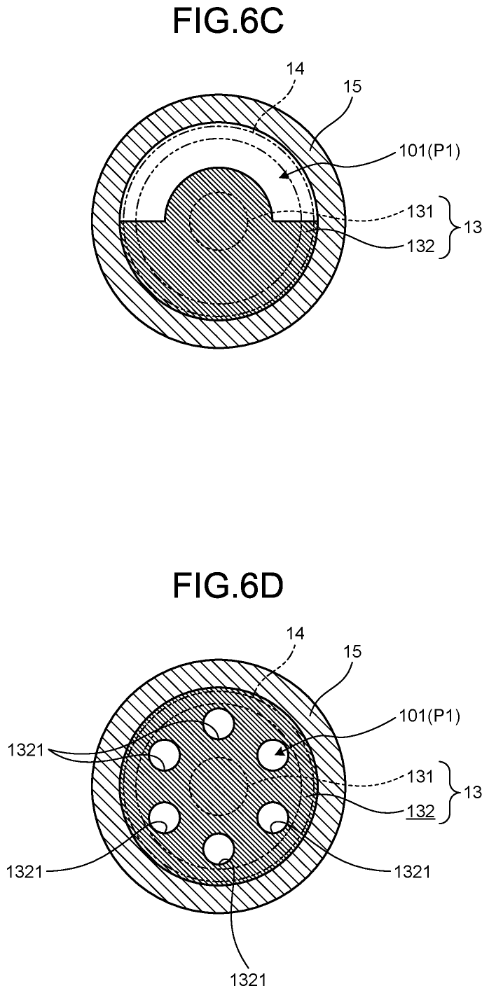

FIGS. 6A to 6D are diagrams illustrating an example of the shape of the flange portion 132. Specifically, FIGS. 6A to 6D are cross-sectional views in which the connecting portion 15 and the flange portion 132 are cut at a position of line A-A illustrated in FIG. 4. It is noted that, in FIGS. 6A to 6D, the tube 14 is illustrated by a two-dot chain line for convenience of explanation.

The flange portion 132 is a portion that is provided at the proximal end of the needle portion body 131 and is fixed to the distal end portion of the second advancing/retreating portion 12. The flange portion 132 is disposed inside the connecting portion 15 and limits a moving distance in the advancing/retreating movement of the needle portion 13. Specifically, the flange portion 132 is configured to come into contact with the proximal end of the knife body 111 when the flange portion 132 moves to the distal end side in the connecting portion 15, and is configured to come into contact with the distal end of the tube 14 when the flange portion 132 moves to the proximal end side. It is noted that the contacting portion when the flange portion 132 moves to the distal end side is not limited to the most proximal end of the knife body 111. A configuration in which a step such as a protrusion is provided in the vicinity of the proximal end of the knife body 111 in the radial direction of the knife body 111 and the flange portion 132 comes into contact with the step when the flange portion 132 moves to the distal end side may be adopted. Similarly, the contacting portion when the flange portion 132 moves to the proximal end side is not limited to the most distal end of the tube 14. A configuration in which a step such as a protrusion is provided in the vicinity of the distal end of the tube 14 in the radial direction of the tube 14 and the flange portion 132 comes into contact with the step when the flange portion 132 moves to the proximal end side may be adopted. In addition, by setting the flange portion 132 to, for example, the shapes illustrated in FIGS. 6A to 6D, the communication state between the passage P1 and the second hole 113 is maintained without blocking the passage P1.

For example, as illustrated in FIG. 6A, the flange portion 132 protrudes from the central axis of the needle portion body 131 in the vertical and horizontal directions in FIG. 6A, and each protruding end portion has a +shape that is slidable on the inner peripheral surface of the connecting portion 15.

In addition, for example, as illustrated in FIG. 6B, the flange portion 132 protrudes from the central axis of the needle portion body 131 in the horizontal directions in FIG. 6A, and each protruding end portion has an I shape that is slidable on the inner peripheral surface of the connecting portion 15.

Furthermore, for example, as illustrated in FIG. 6C, the flange portion 132 is formed in a semicircular shape whose center coincides with the central axis of the needle portion body 131. An arc portion of the outer edge of the flange portion 132 is slidable on the inner peripheral surface of the connecting portion 15.

In addition, for example, as illustrated in FIG. 6D, the flange portion 132 may be formed in a circular shape whose center coincides with the central axis of the needle portion body 131, and has a plurality of through holes 1321 penetrating along the central axis thereof. An arc portion of the outer edge of the flange portion 132 is slidable on the inner peripheral surface of the connecting portion 15.

It is noted that the flange portion 132 may be integrally formed with the needle portion body 131, and the flange portion 132 and the needle portion body 131 may be formed separately and may be fixed to each other by bonding or the like. In addition, the flange portion 132 may be integrally formed with the wire 12, and the flange portion 132 and the wire 12 may be formed separately and may be fixed to each other by bonding or the like.

The knife 11 and the needle portion 13 described above adopt the following structures in order to avoid conduction with each other.

For example, insulating coating is performed on at least one of the inner peripheral surface of the second hole 113 or the front surface of the needle portion 13.

In addition, for example, the needle portion 13 is made of a material having electrical insulating properties.

Operation of Treatment Tool for Endoscope

Next, the operation of the treatment tool 6 for an endoscope described above will be described. Hereinafter, for convenience of explanation, the flow of ESD will be described as an example.

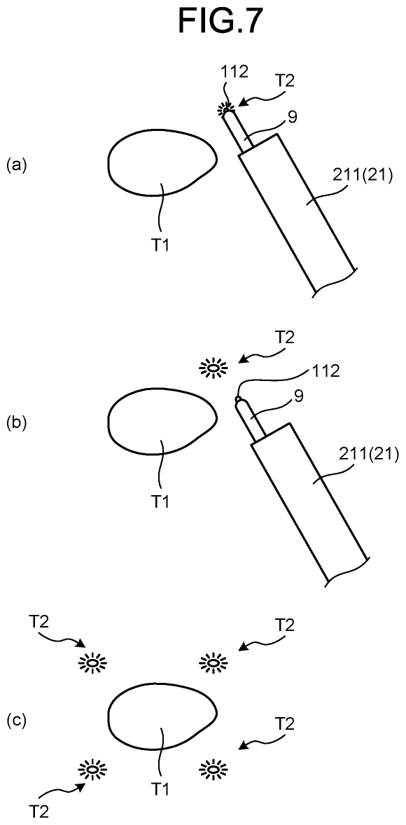

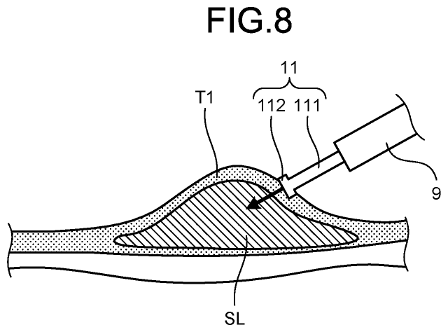

FIGS. 7 to 10 are diagrams for describing the operation of the treatment tool 6 for an endoscope. Specifically, FIG. 7 is a diagram for describing a marking step in ESD. FIGS. 8 to 10 are diagrams for describing a local injection step in ESD.

First, an operator such as an operating surgeon inserts the endoscope insertion portion 21 into the body cavity and moves the distal end of the endoscope insertion portion 21 to the vicinity of the target site T1 (FIG. 7).

Next, the operator such as the operating surgeon performs a first retreating operation of pulling the first slider 82 toward the front (ring 811 side). Due to this, the treatment tool insertion portion 7 is in a state where the protrusion portion 112 is located in the large diameter portion 9211, the proximal end of the protrusion portion 112 comes into contact with the first stepped portion 9213, and only the protrusion portion 112 protrudes from the first hole 921 to the outside of the distal end part 92, that is, a state (corresponding to a second mode) where the protrusion portion 112 protrudes from the first hole 921 and the knife body 111 is located in the first hole 921. Next, the operator such as the operating surgeon performs a second retreating operation of pulling the second slider 83 toward the front (ring 811 side). Due to this, the treatment tool insertion portion 7 is in a state (corresponding to a fourth mode) where the flange portion 132 comes into contact with the distal end of the tube 14 and the distal end of the needle portion 13 is located in the second hole 113.

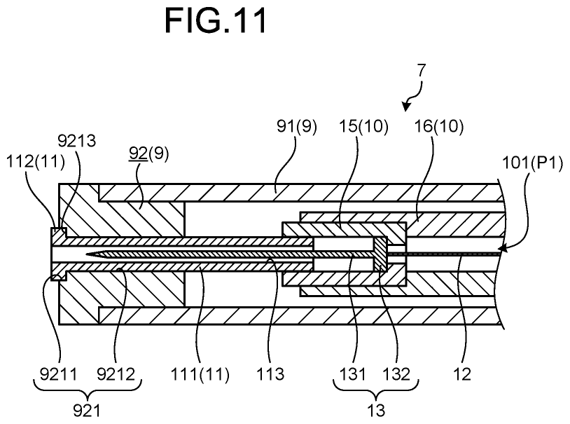

FIG. 11 is a diagram illustrating an example of a configuration in a case where the tube 14 is not provided. Specifically, FIG. 11 is a cross-sectional view corresponding to FIG. 2.

It is noted that, as illustrated in FIG. 11, in a case where the tube 14 is not provided, instead of the tube 14, the inner diameter of the proximal end portion of the connecting portion 15 is made smaller than the maximum width of the flange portion 132, and thus, the flange portion 132 may abut on the proximal end portion of the connecting portion 15 when the second retreating operation is performed.

As described above, the treatment tool insertion portion 7 is in the first state illustrated in FIG. 2 by the first and second retreating operations on the first and second sliders 82 and 83. An operator such as an operating surgeon inserts the treatment tool insertion portion 7 in the first state from the insertion port 223 into the pipeline inside the endoscope insertion portion 21 and protrudes the treatment tool insertion portion 7 from the distal end of the endoscope insertion portion 21. It is noted that, even when the knife 11 slightly protrudes from the distal end part 92, there is no problem in inserting the treatment tool insertion portion 7 in the first state from the insertion port 223 into the pipeline inside the endoscope insertion portion 21.

Next, the operator such as the operating surgeon performs a marking step as shown below.

That is, the operator such as the operating surgeon operates the operating portion (not illustrated) such as a foot switch while maintaining the first state of the treatment tool insertion portion 7 by the first and second retreating operations on the first and second sliders 82 and 83, and energizes the knife 11 with a high frequency current from the power source 100. As illustrated in (a) of FIG. 7, the operator such as the operating surgeon presses the protrusion portion 112 against a living tissue (mucosal surface) around the target site T1. Due to this, the living tissue coming into contact with the protrusion portion 112 is cauterized. That is, as illustrated in (a) of FIG. 7 or (b) of FIG. 7, a marking sign T2 is formed on the cauterized portion.

The operator such as the operating surgeon repeats the above-described operation a plurality of times to form a number of marking signs T2 so that the outer edge of the target site T1 can be grasped, as illustrated in (c) of FIG. 7. After that, the operator such as the operating surgeon ends the energization of the knife 11 with the high frequency current from the power source 100.

Next, the operator such as the operating surgeon performs a local injection step as shown below.

That is, the operator such as the operating surgeon performs a first advancing operation of pushing the first slider 82. Due to this, the treatment tool insertion portion 7 is in a state (corresponding to a first mode) where the distal end of the connecting portion 15 comes into contact with the proximal end of the distal end part 92 and the knife 11 protrudes from the distal end of the sheath 9 by the maximum protrusion length. Next, the operator such as the operating surgeon performs a second advancing operation of pushing the second slider 83. Due to this, the treatment tool insertion portion 7 is in a state (corresponding to a third mode) where the flange portion 132 comes into contact with the proximal end of the knife body 111 and the needle portion 13 protrudes from the distal end of the knife 11 by the maximum protrusion length (for example, 1 mm).

As described above, the treatment tool insertion portion 7 is in the second state illustrated in FIG. 3 by the first and second advancing operations on the first and second sliders 82 and 83. In the second state, the operator such as the operating surgeon can make a cut in the living tissue from the mucosal surface to the submucosal layer by piercing the distal end of the needle portion 13 into the living tissue around the target site T1.

That is, the operator such as the operating surgeon operates the operating portion (not illustrated) such as a foot switch while maintaining the second state of the treatment tool insertion portion 7 by the first and second advancing operations on the first and second sliders 82 and 83, and supplies the physiological saline solution from the water supply source 200. Due to this, the physiological saline solution supplied from the water supply source 200 is discharged from the distal end of the knife 11 after following the passage P1 to the passage of the second hole 113. The discharged physiological saline solution SL is injected into the submucosal layer below the target site T1 (FIG. 8). The target site T1 (mucosal surface) bulges (floats) from other tissues such as the submucosal layer therebelow.

It is noted that, when injecting the physiological saline solution SL below the target site T1, the following two methods can be considered.

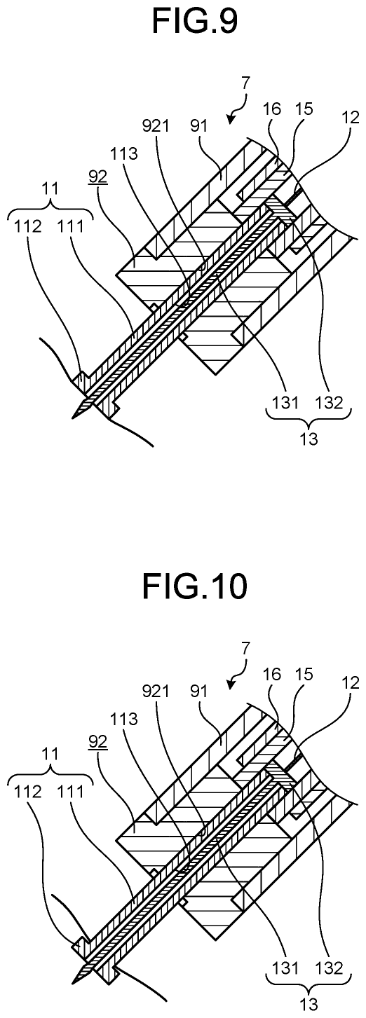

As illustrated in FIG. 9, the first method is a method of injecting the physiological saline solution SL below the target site T1 in a state where the distal end of the needle portion 13 is inserted into the living tissue.

As illustrated in FIG. 10, the second method is a method of injecting the physiological saline solution SL below the target site T1 in a state where the protrusion portion 112 is inserted into the living tissue together with the distal end of the needle portion 13.

Next, the operator such as the operating surgeon performs an incision step as shown below.

That is, the operator such as the operating surgeon performs a first advancing operation on the first slider 82 and performs a second retreating operation on the second slider 83. When the first advancing operation is performed on the first slider 82, as described above, the treatment tool insertion portion 7 is in a state where the knife 11 protrudes from the distal end of the sheath 9 by the maximum protrusion length. In addition, when the second retreating operation is performed on the second slider 83, as described above, the treatment tool insertion portion 7 is in a state where the distal end of the needle portion 13 is located in the second hole 113.

As described above, the treatment tool insertion portion 7 is in the third state illustrated in FIG. 4 by the first advancing operation on the first slider 82 and the second retreating operation on the second slider 83.

In addition, the operator such as the operating surgeon operates the operating portion (not illustrated) such as a foot switch while maintaining the third state of the treatment tool insertion portion 7 by the first advancing operation on the first slider 82 and the second retreating operation on the second slider 83, and energizes the knife 11 with a high frequency current from the power source 100. The operator such as the operating surgeon moves the protrusion portion 112 along the periphery of the target site T1 in a state where the living tissue is pierced while confirming the marking sign T2, and incises the entire periphery of the target site T1.

After that, while maintaining the third state, the submucosal layer including the target site T1, the entire periphery of which is incised, is removed by performing dissection or the like on the submucosal layer. In the third state, since the distal end of the needle portion 13 is located in the second hole 113, the incision or dissection performance of the knife 11 is not affected.

ESD is completed by the above steps.

As described above, the needle portion 13 according to the first embodiment can advance and retreat from the position where the distal end is housed inside the knife 11 to the position where the distal end protrudes from the second hole 113. In other words, the distal end of the needle portion 13 is housed inside the knife 11 in a retracted state. The distal end of the needle portion 13 protrudes from the distal end of the knife 11 in an extended state.

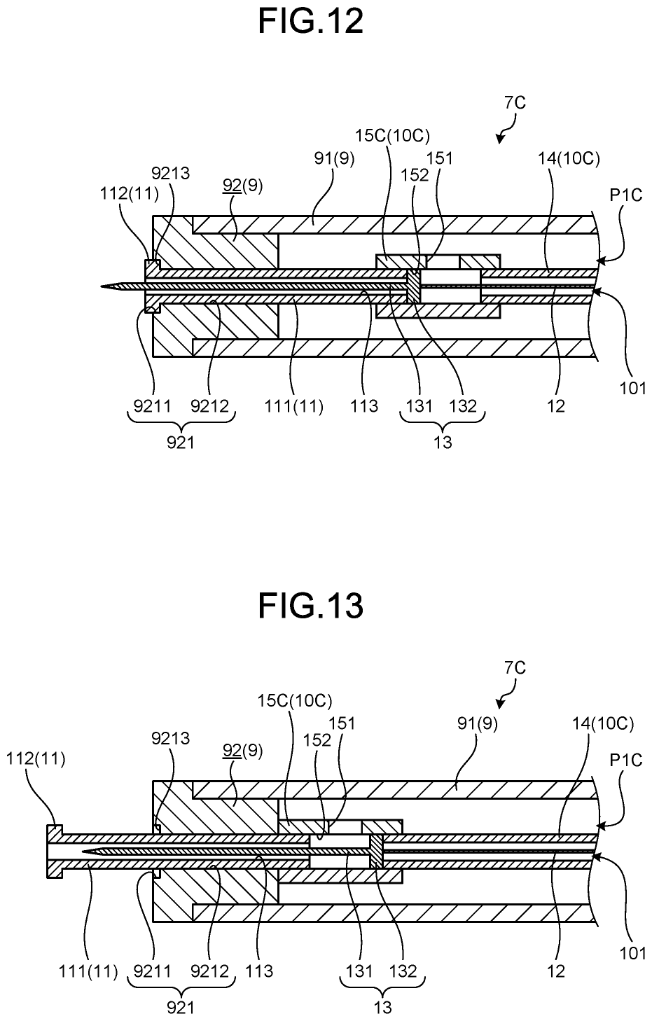

Hereinafter, a modification of the first embodiment will be described with reference to FIGS. 12 and 13 by taking a treatment tool insertion portion 7C, which is a configuration of a fourth embodiment described below, as an example. It is noted that the detailed configuration of the treatment tool insertion portion 7C (the passage P1C, a first advancing/retreating portion 10C, and a connecting portion 15C) will be described in a fourth embodiment described below.

In the first embodiment, an example in which the needle portion 13 is made of a material having electrical insulating properties is shown, but the disclosure is not limited thereto. For example, the needle portion 13 may be made of an energizable material such as a metal. When the needle portion 13 is made of an energizable material, since the outer edge (outer peripheral surface) of the flange portion 132 is always in contact with the connecting portion 15C, the needle portion 13 can be energized via the connecting portion 15C without depending on the amount of protrusion from the knife body 111. In addition, the method of energizing the needle portion 13 is not limited thereto. By performing insulating coating on the outer edge (outer peripheral surface) of the flange portion 132 or the inner peripheral surface of a communication hole 152, the proximal end portion of the knife body 111 and the flange portion 132 may come into contact with each other to energize the needle portion 13 when the needle portion 13 protrudes from the knife body 111 to the maximum extent.

When the tissue is incised with the needle portion 13, the distal end portion of the needle portion 13 is moved to the position protruding from the distal end of the knife 11 while retreating the distal end (protrusion portion 112) of the knife 11 to the position that abuts on the distal end of the sheath 9. As illustrated in FIG. 12, when the distal end portion of the needle portion 13 protrudes from the distal end of the knife 11, the flange portion 132 may abut on a portion of the knife 11. In this state, the tissue can be incised at the distal end portion of the needle portion 13. In addition, the puncturing of the distal end of the needle portion 13 into the tissue for local injection and the incision of the tissue may be alternately repeated. Furthermore, the incision of the tissue by the needle portion 13 and the incision of the tissue by the knife 11 may be alternately repeated. For example, after the tissue is incised with the needle portion 13, as illustrated in FIG. 13, the incision of the tissue may be performed with the knife 11 in a state where the needle portion 13 is housed inside the knife 11 and the distal end of the knife 11 is located on a distal side rather than the distal end of the sheath 9 and the needle portion 13. Here, the order of the incision by the needle portion 13 and the incision by the knife 11 may be changed as appropriate. It is noted that the incision of the tissue includes not only the incision or excision of the mucosa but also the dissection of the submucosal layer.

According to the first embodiment described above, the following effects are obtained.

In the treatment tool 6 for an endoscope according to the first embodiment, the treatment tool insertion portion 7 includes the needle portion 13 that makes a cut in the living tissue inside the body cavity, and the knife 11 that injects the physiological saline solution into the living tissue through the cut.

Therefore, in the treatment tool 6 for an endoscope according to the first embodiment, local injection can be performed satisfactorily by passing through the cut.

In particular, in the treatment tool 6 for an endoscope according to the first embodiment, in ESD, the local injection step and other steps can be performed by the single treatment tool 6 for an endoscope, without changing the treatment tool between the local injection step and other steps. Therefore, convenience can be improved.

In addition, in the treatment tool 6 for an endoscope according to the first embodiment, the needle portion 13 is a solid body. Therefore, compared to the configuration in which the needle portion 13 is provided with a hole for local injection, the needle portion 13 does not buckle and the life of the treatment tool insertion portion 7 can be extended.

In addition, in the treatment tool 6 for an endoscope according to the first embodiment, the needle portion 13 and the second advancing/retreating portion 12 are disposed in the second hole 113 and the communication hole 101. Therefore, the first advancing/retreating portion 10, the knife 11, the second advancing/retreating portion 12, and the needle portion 13 can be compactly assembled and the diameter of the treatment tool insertion portion 7 can be reduced.

### Second Embodiment

Next, the second embodiment will be described.

In the following description, the same reference numerals are assigned to the same components as those in the first embodiment described above and detailed description thereof will be omitted or simplified.

The treatment tool 6 for an endoscope according to the second embodiment differs from the treatment tool 6 for an endoscope described above in the first embodiment in the configuration of the distal end portion of the treatment tool insertion portion 7. Hereinafter, for convenience of explanation, the treatment tool insertion portion according to the second embodiment is referred to as a treatment tool insertion portion 7A.

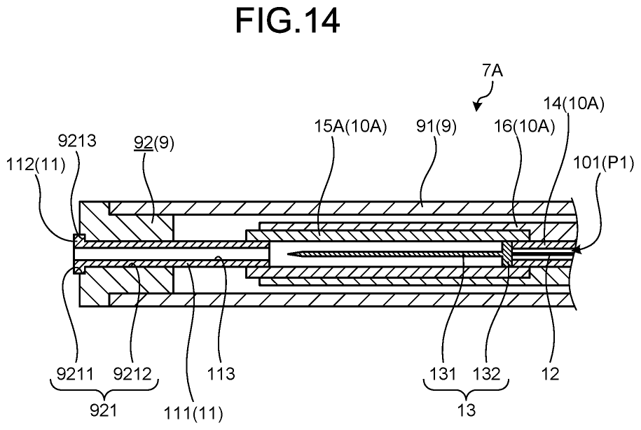

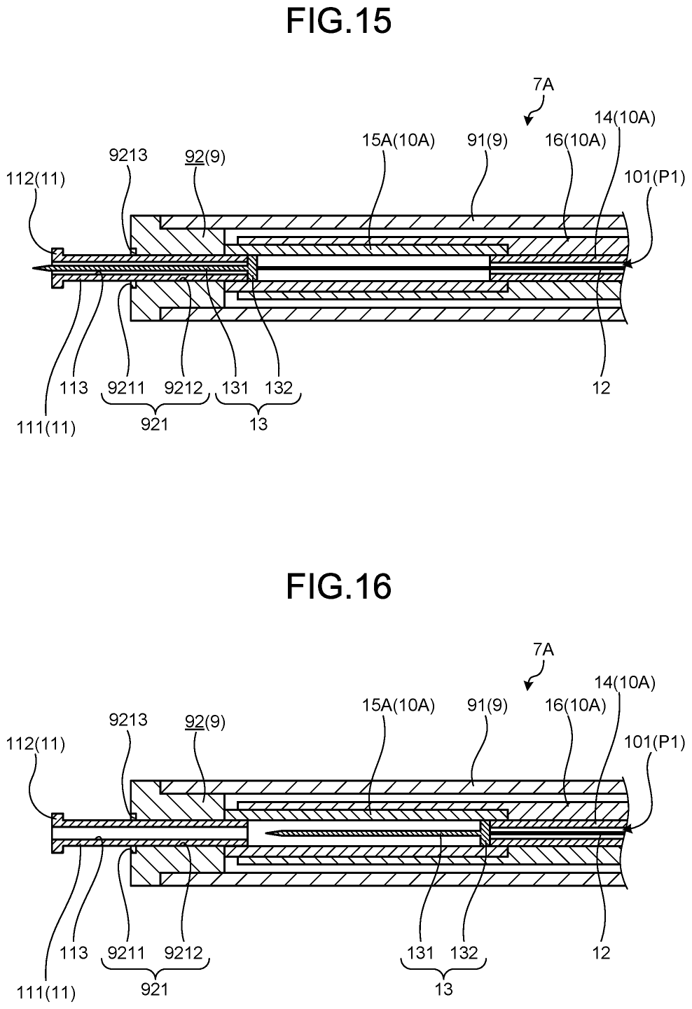

FIGS. 14 to 16 are diagrams for describing the configuration of the treatment tool insertion portion 7A according to the second embodiment. Specifically, FIG. 14 is a cross-sectional view corresponding to FIG. 2 and is a diagram illustrating the treatment tool insertion portion 7A set to a first state. FIG. 15 is a cross-sectional view corresponding to FIG. 3 and is a diagram illustrating the treatment tool insertion portion 7A set to a second state. FIG. 16 is a cross-sectional view corresponding to FIG. 4 and is a diagram illustrating the treatment tool insertion portion 7A set to a third state.

As illustrated in FIGS. 14 to 16, the treatment tool insertion portion 7A differs from the treatment tool insertion portion 7 described above in the first embodiment in that the total length of the connecting portion 15 in the longitudinal direction is lengthened. Hereinafter, for convenience of explanation, a first advancing/retreating portion and a connecting portion 15 according to the second embodiment are referred to as a first advancing/retreating portion 10A and a connecting portion 15A, respectively.

In the second embodiment, the treatment tool insertion portion 7A is in the first state shown below by the first and second retreating operations on first and second sliders 82 and 83.

Specifically, due to the first retreating operation on the first slider 82, as illustrated in FIG. 14, similar to the first embodiment described above, the treatment tool insertion portion 7A is in a state where a protrusion portion 112 is located inside a large diameter portion 9211, a proximal end of the protrusion portion 112 comes into contact with a first stepped portion 9213, and only the protrusion portion 112 protrudes from a first hole 921 to the outside of a distal end part 92. In addition, due to the second retreating operation on the second slider 83, the treatment tool insertion portion 7A is in a state where a flange portion 132 comes into contact with a distal end of a tube 14 and an entire needle portion 13 is located inside the connecting portion 15A.

In addition, in the second embodiment, the treatment tool insertion portion 7A is in the second state shown below by the first and second advancing operations on the first and second sliders 82 and 83.

Specifically, due to the first advancing operation on the first slider 82, as illustrated in FIG. 15, similar to the first embodiment described above, the treatment tool insertion portion 7A is in a state where the distal end of the connecting portion 15A comes into contact with the proximal end of the distal end part 92 and a knife 11 protrudes from a distal end of a sheath 9 by the maximum protrusion length. In addition, due to the second advancing operation on the second slider 83, similar to the first embodiment described above, the treatment tool insertion portion 7A is in a state where the flange portion 132 comes into contact with a proximal end of a knife body 111 and the needle portion 13 protrudes from the distal end of the knife 11 by the maximum protrusion length.

Furthermore, in the second embodiment, due to the first advancing operation on the first slider 82 and the second retreating operation on the second slider 83, the treatment tool insertion portion 7A is in the third state shown below.

Specifically, due to the first advancing operation on the first slider 82, as illustrated in FIG. 16, as described above, the treatment tool insertion portion 7A is in a state where the distal end of the connecting portion 15A comes into contact with the proximal end of the distal end part 92 and the knife 11 protrudes from the distal end of the sheath 9 by the maximum protrusion length. In addition, due to the second retreating operation on the second slider 83, as described above, the treatment tool insertion portion 7A is in a state where the flange portion 132 comes into contact with the distal end of the tube 14 and the entire needle portion 13 is located inside the connecting portion 15A.

It is noted that, since the operation of the treatment tool 6 for an endoscope according to the second embodiment is the same as the operation of the first embodiment described above, the description thereof will be omitted.

As described above, the needle portion 13 according to the second embodiment can advance and retreat from the position where the distal end is housed inside the first advancing/retreating portion 10A to the position where the distal end protrudes from the second hole 113.

Even when the treatment tool insertion portion 7A according to the second embodiment described above is adopted, the same effects as those of the first embodiment described above can be obtained.

In addition, in the treatment tool insertion portion 7A according to the second embodiment, the second advancing/retreating portion 12 advances and retreats the needle portion 13 from the position where the entire needle portion 13 is located on the proximal end side of the second hole 113 to the position where the distal end side of the needle portion 13 protrudes from the second hole 113 toward the distal end side.

Therefore, when the physiological saline solution is supplied from the water supply source 200 while the entire needle portion 13 is located inside the connecting portion 15A by the second retreating operation on the second slider 83, surgical site cleaning can be performed during each step of ESD, as shown below.

That is, in a state where the entire needle portion 13 is located inside the connecting portion 15A, the area of the passage of the physiological saline solution flowing through the second hole 113 is large, compared to a state where the needle portion body 131 is located inside the second hole 113. Therefore, when the physiological saline solution is supplied from the water supply source 200 while the entire needle portion 13 is located inside the connecting portion 15A by the second retreating operation on the second slider 83, the water pressure of the physiological saline solution discharged from the distal end of the knife 11 is relatively low. That is, the surgical site can be cleaned by supplying the physiological saline solution to the surgical site.

### Third Embodiment

Next, the third embodiment will be described.

In the following description, the same reference numerals are assigned to the same components as those in the second embodiment described above and detailed description thereof will be omitted or simplified.

The treatment tool 6 for an endoscope according to the third embodiment differs from the treatment tool 6 for an endoscope described above in the second embodiment in the configuration of the distal end portion of the treatment tool insertion portion 7A. Hereinafter, for convenience of explanation, the treatment tool insertion portion according to the third embodiment is referred to as a treatment tool insertion portion 7B.

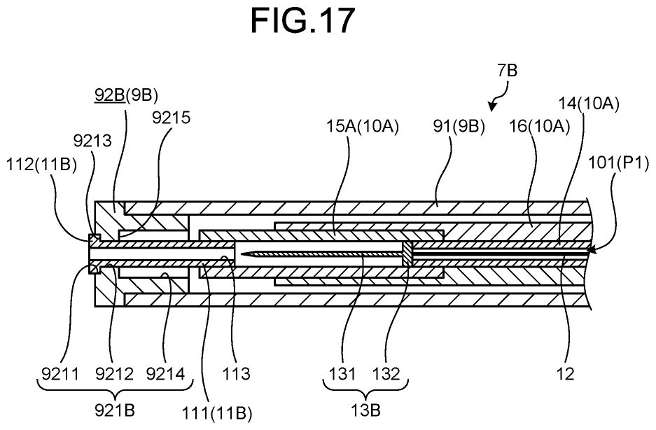

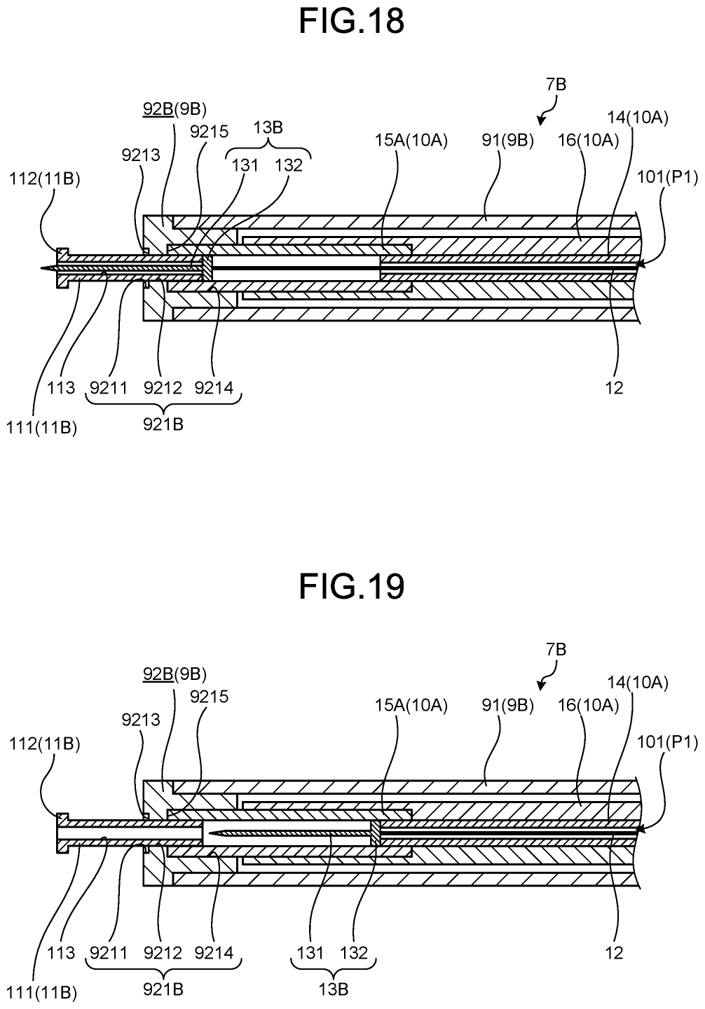

FIGS. 17 to 19 are diagrams for describing the configuration of the treatment tool insertion portion 7B according to the third embodiment. Specifically, FIG. 17 is a cross-sectional view corresponding to FIG. 14 and is a diagram illustrating the treatment tool insertion portion 7B set to a first state. FIG. 18 is a cross-sectional view corresponding to FIG. 15 and is a diagram illustrating the treatment tool insertion portion 7B set to a second state. FIG. 19 is a cross-sectional view corresponding to FIG. 16 and is a diagram illustrating the treatment tool insertion portion 7B set to a third state.

As illustrated in FIGS. 17 to 19, the treatment tool insertion portion 7B differs from the treatment tool insertion portion 7A described above in the second embodiment in that the total length of the knife 11 and the needle portion 13 in the longitudinal direction is shortened. Hereinafter, for convenience of explanation, the knife and the needle portion according to the third embodiment are referred to as a knife 11B and a needle portion 13B respectively.

In addition, as illustrated in FIGS. 17 to 19, the treatment tool insertion portion 7B differs from the treatment tool insertion portion 7A described above in the second embodiment in the shape of the distal end part 92 of the sheath 9. Hereinafter, for convenience of explanation, the sheath and the distal end part according to the third embodiment are referred to as a sheath 9B and a distal end part 92B, respectively.

As illustrated in FIGS. 17 to 19, the distal end part 92B differs from the distal end part 92 described above in the second embodiment in the shape of the first hole 921. Hereinafter, for convenience of explanation, the first hole according to the third embodiment is referred to as a first hole 921B.

As illustrated in FIGS. 17 to 19, the first hole 921B includes a storage hole 9214 in addition to a large diameter portion 9211 and a small diameter portion 9212 similar to the first hole 921 described above in the second embodiment.

The storage hole 9214 is located on the proximal end side of the small diameter portion 9212. The inner diameter dimension of the storage hole 9214 is set to be larger than that of the small diameter portion 9212 and slightly larger than that of the connecting portion 15A. In the following, for convenience of explanation, a stepped portion between the small diameter portion 9212 and the storage hole 9214 is referred to as a second stepped portion 9215. It is noted that the total length of the knife 11B and the needle portion 13B in the longitudinal direction is shorter than the total length of the knife 11 and the needle portion 13 described above in the second embodiment by the total length of the storage hole 9214 in the longitudinal direction.

In the third embodiment, the treatment tool insertion portion 7B is in the first state shown below by the first and second retreating operations on first and second sliders 82 and 83.

Specifically, due to the first retreating operation on the first slider 82, as illustrated in FIG. 17, similar to the second embodiment described above, the treatment tool insertion portion 7B is in a state where a protrusion portion 112 is located inside a large diameter portion 9211, a proximal end of the protrusion portion 112 comes into contact with a first stepped portion 9213, and only the protrusion portion 112 protrudes from the first hole 921B to the outside of the distal end part 92B. At this time, the connecting portion 15A is located on the proximal end side of the storage hole 9214. In addition, due to the second retreating operation on the second slider 83, the treatment tool insertion portion 7B is in a state where a flange portion 132 comes into contact with a distal end of a tube 14 and the entire needle portion 13B is located inside the connecting portion 15A.

In addition, in the third embodiment, the treatment tool insertion portion 7B is in the second state shown below by the first and second advancing operations on the first and second sliders 82 and 83.

Specifically, due to the first advancing operation on the first slider 82, as illustrated in FIG. 18, the treatment tool insertion portion 7B is in a state where the distal end portion of the connecting portion 15A is located inside the storage hole 9214, the distal end of the connecting portion 15A comes into contact with the second stepped portion 9215, and the knife 11B protrudes from the distal end of the sheath 9B by the maximum protrusion length. In addition, due to the second advancing operation on the second slider 83, similar to the second embodiment described above, the treatment tool insertion portion 7B is in a state where the flange portion 132 comes into contact with a proximal end of a knife body 111 and the needle portion 13B protrudes from the distal end of the knife 11B by the maximum protrusion length (for example, 1 mm, etc.).

Furthermore, in the third embodiment, due to the first advancing operation on the first slider 82 and the second retreating operation on the second slider 83, the treatment tool insertion portion 7B is in the third state shown below.

Specifically, due to the first advancing operation on the first slider 82, as illustrated in FIG. 19, as described above, the treatment tool insertion portion 7B is in a state where the distal end portion of the connecting portion 15A is located inside the storage hole 9214, the distal end of the connecting portion 15A comes into contact with the second stepped portion 9215, and the knife 11B protrudes from the distal end of the sheath 9B by the maximum protrusion length. In addition, due to the second retreating operation on the second slider 83, as described above, the treatment tool insertion portion 7B is in a state where the flange portion 132 comes into contact with the proximal end of the knife body 111 and the needle portion 13B protrudes from the distal end of the knife 11B by the maximum protrusion length.

It is noted that, since the operation of the treatment tool 6 for an endoscope according to the third embodiment is the same as the operation of the second embodiment described above, the description thereof will be omitted.

Even when the treatment tool insertion portion 7B according to the third embodiment described above is adopted, the same effects as those of the second embodiment described above can be obtained.

In addition, in the treatment tool insertion portion 7B according to the third embodiment, the total length of the knife 11B and the needle portion 13B in the longitudinal direction is short, compared to the treatment tool insertion portion 7A described above in the second embodiment. Therefore, when the treatment tool insertion portion 7B is inserted from the insertion port 223 into the pipeline of the endoscope insertion portion 21, the treatment tool insertion portion 7B can be easily bent into a shape along the pipeline, and the insertability can be improved.

### Fourth Embodiment

Next, the fourth embodiment will be described. In the following description, the same reference numerals are assigned to the same components as those in the first embodiment described above and detailed description thereof will be omitted or simplified.

The treatment tool 6 for an endoscope according to the fourth embodiment differs from the treatment tool 6 for an endoscope described above in the first embodiment in the configuration of the distal end portion of the treatment tool insertion portion 7. Hereinafter, for convenience of explanation, the treatment tool insertion portion according to the fourth embodiment is referred to as a treatment tool insertion portion 7C.

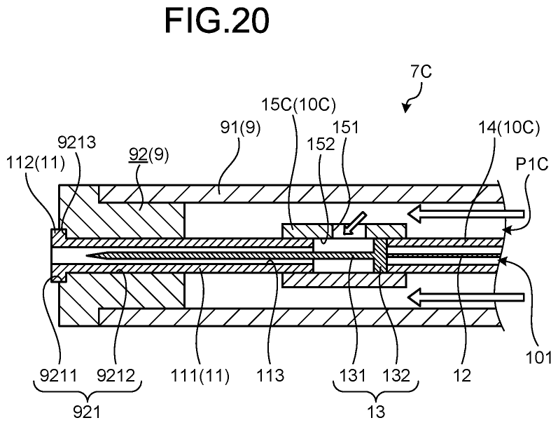

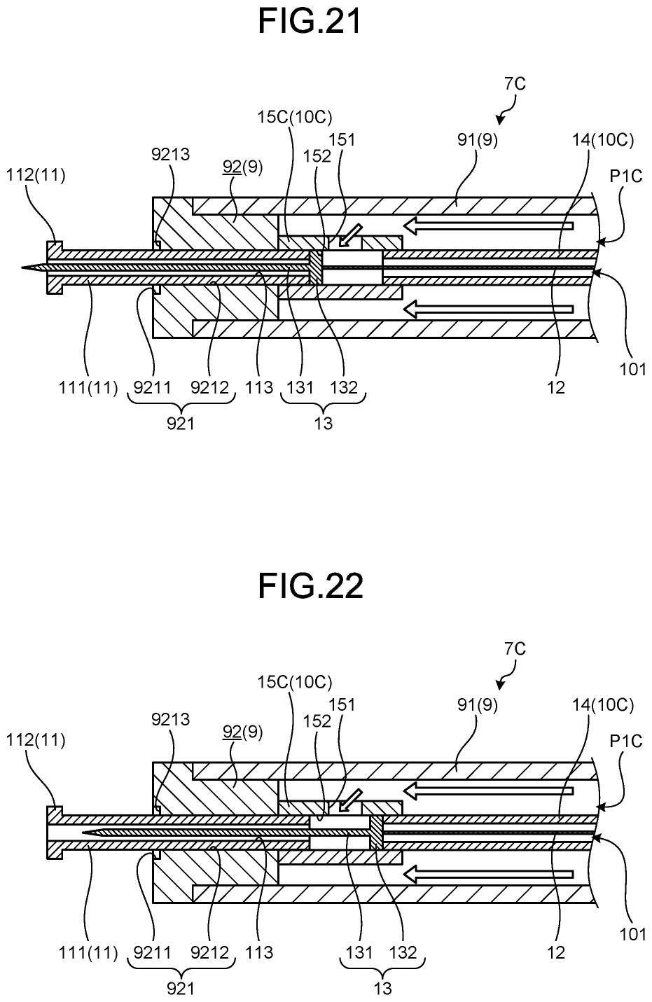

FIGS. 20 to 22 are diagrams for describing the configuration of the treatment tool insertion portion 7C according to the fourth embodiment. Specifically, FIG. 20 is a cross-sectional view corresponding to FIG. 2 and is a diagram illustrating the treatment tool insertion portion 7C set to a first state. FIG. 21 is a cross-sectional view corresponding to FIG. 3 and is a diagram illustrating the treatment tool insertion portion 7C set to a second state. FIG. 22 is a cross-sectional view corresponding to FIG. 4 and is a diagram illustrating the treatment tool insertion portion 7C set to a third state.

In addition, as illustrated in FIGS. 20 to 22, the treatment tool insertion portion 7C differs from the treatment tool insertion portion 7 described above in the first embodiment in that the inner tube 16 is omitted and differs in the passage P1 through which the physiological saline solution flows and the shape of the connecting portion 15. Hereinafter, for convenience of explanation, a passage, a first advancing/retreating portion, and a connecting portion according to the fourth embodiment are referred to as a passage P1C, a first advancing/retreating portion 10C, and a connecting portion 15C, respectively.

An inside of a sheath body 91 according to the fourth embodiment communicates with a water supply port 812. That is, a space between the inner peripheral surface of the sheath body 91, the outer peripheral surface of the tube 14, and the outer peripheral surface of the connecting portion 15C functions as the passage P1C, through which the physiological saline solution supplied from a water supply source 200 flows via the tube TU and the water supply port 812.

As illustrated in FIGS. 20 to 22, a communication hole 151 that penetrates the inside and outside of the connecting portion 15C is provided on the outer peripheral surface of the connecting portion 15C.

The inside of the connecting portion 15C corresponds to a first communication hole 152 (FIGS. 20 to 22), which communicates with a second hole 113. In addition, the communication hole 151 corresponds to a second communication hole, which communicates with the passage P1C and the first communication hole 152.

It is noted that the operation of the treatment tool 6 for an endoscope according to the fourth embodiment differs from the first embodiment described above only in the passage of the physiological saline solution in the treatment tool insertion portion 7C (see the arrows illustrated in FIGS. 20 and 21), and thus, the description thereof is omitted.

Even when the treatment tool insertion portion 7C according to the fourth embodiment described above is adopted, the same effects as those of the first embodiment described above can be obtained.

In addition, in the treatment tool insertion portion 7C according to the fourth embodiment, the inner tube 16 can be omitted. Therefore, the configuration of the treatment tool insertion portion 7C can be simplified, and the diameter of the treatment tool insertion portion 7C can be reduced.

### Other Embodiments

Although the modes for carrying out the disclosure have been described above, the disclosure should not be limited only to the first to fourth embodiments described above.

In the first to fourth embodiments described above, the shape of the protrusion portion 112 is not limited to the shapes described above in the first to fourth embodiments, and other shapes may also be adopted. For example, the knives 11 and 11B may be configured by a so-called hook knife.

In the first to fourth embodiments described above, the knives 11 and 11B are configured to be able to advance and retreat, but the disclosure is not limited thereto, and the knives 11 and 11B may be unable to advance and retreat. That is, the configuration in which the knives 11 and 11B are always in any of the following states (1) and (2) may be adopted.

(1) The knives 11 and 11B are always in a state of protruding from the distal ends of the sheaths 9 and 9B by the maximum protrusion length (for example, the state of FIG. 2).

(2) The knives 11 and 11B are always in a state in which only the protrusion portion 112 protrudes from the first holes 921 and 921B to the outside of the distal end parts 92 and 92B (for example, the state of FIG. 3).

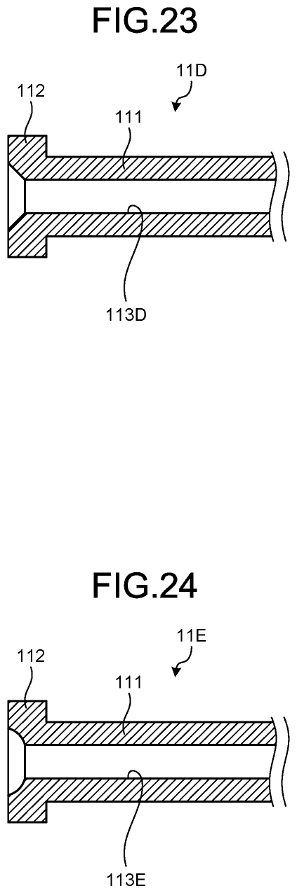

FIG. 23 is a diagram illustrating a first modification of the first to fourth embodiments.

In the first modification, as illustrated in FIG. 23, the shape of the second hole 113 described above in the first to fourth embodiments is different. Hereinafter, for convenience of explanation, a knife and a second hole according to the first modification are referred to as a knife 11D and a second hole 113D, respectively. It is noted that FIG. 23 is a cross-sectional view in which the knife 11D according to the first modification is cut by a cutting plane along the central axis of the knife 11D.

As illustrated in FIG. 23, the edge portion of the distal end of the second hole 113D is chamfered linearly in a cross-section. Since the second hole 113D is formed as described above, when needle portions 13 and 13B protruding from the second hole 113D toward the distal end side are pulled toward the proximal end side, the needle portions 13 and 13B are not caught in the edge portion of the distal end of the second hole 113D. Therefore, a second advancing operation and a second retreating operation can be performed smoothly.

FIG. 24 is a diagram illustrating a second modification of the first to fourth embodiments.

In the second modification, as illustrated in FIG. 24, the shape of the second hole 113 described above in the first to fourth embodiments is different. Hereinafter, for convenience of explanation, a knife and a second hole according to the second modification are referred to as a knife 11E and a second hole 113E, respectively. It is noted that FIG. 24 is a cross-sectional view in which the knife 11E according to the second modification is cut by a cutting plane along the central axis of the knife 11E.

As illustrated in FIG. 24, the edge portion of the distal end of the second hole 113E is chamfered linearly in a cross-section. Since the second hole 113E is formed as described above, when needle portions 13 and 13B protruding from the second hole 113E toward the distal end side are pulled toward the proximal end side, the needle portions 13 and 13B are not caught in the edge portion of the distal end of the second hole 113E. Therefore, a second advancing operation and a second retreating operation can be performed smoothly.

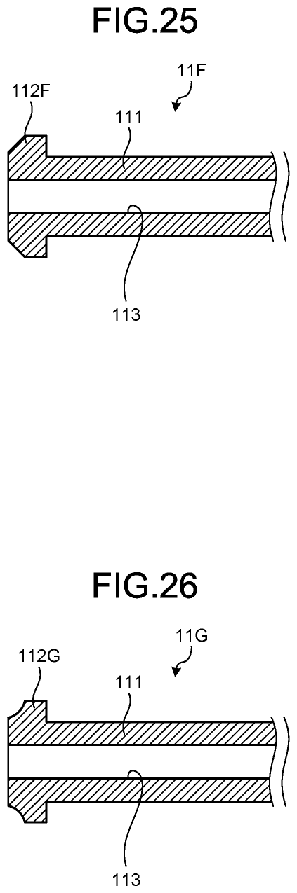

FIG. 25 is a diagram illustrating a third modification of the first to fourth embodiments.

In the third modification, as illustrated in FIG. 25, the shape of the protrusion portion 112 described above in the first to fourth embodiments is different. Hereinafter, for convenience of explanation, a knife and a protrusion portion according to the third modification are referred to as a knife 11F and a protrusion portion 112F, respectively. It is noted that FIG. 25 is a cross-sectional view in which the knife 11F according to the third modification is cut by a cutting plane along the central axis of the knife 11F.

As illustrated in FIG. 25, the edge portion of the outer peripheral edge of the distal end of the protrusion portion 112F is chamfered linearly in a cross-section. Since the protrusion portion 112F is formed as described above, when the protrusion portion 112F is inserted into the living tissue as described with reference to FIG. 10, the insertion can be performed smoothly.

FIG. 26 is a diagram illustrating a fourth modification of the first to fourth embodiments.

In the fourth modification, as illustrated in FIG. 26, the shape of the protrusion portion 112 described above in the first to fourth embodiments is different. Hereinafter, for convenience of explanation, a knife and a protrusion portion according to the fourth modification are referred to as a knife 11G and a protrusion portion 112G, respectively. It is noted that FIG. 26 is a cross-sectional view in which the knife 11G according to the fourth modification is cut by a cutting plane along the central axis of the knife 11G.

As illustrated in FIG. 26, the edge portion of the outer peripheral edge of the distal end of the protrusion portion 112G is chamfered in a curved shape in a cross-section. Since the protrusion portion 112G is formed as described above, when the protrusion portion 112G is inserted into the living tissue as described with reference to FIG. 10, the insertion can be performed smoothly.

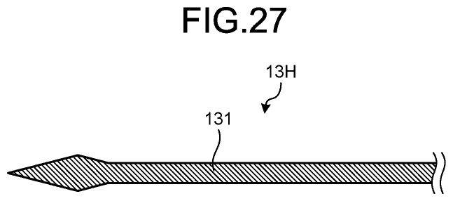

FIG. 27 is a diagram illustrating a fifth modification of the first to fourth embodiments.

In the fifth modification, as illustrated in FIG. 27, the shape of the needle portions 13 and 13B described above in the first to fourth embodiments is different. Hereinafter, for convenience of explanation, the needle portion according to the fifth embodiment is referred to as a needle portion 13H. It is noted that FIG. 27 is a cross-sectional view in which the needle portion 13H according to the fifth modification is cut by a cutting plane along the central axis of the needle portion 13H.

The outer diameter dimension of the distal end portion of the needle portion 13H increases toward the distal end and gradually decreases further toward the distal end. Since the needle portion 13H is formed as described above, similar to the first to fourth embodiments described above, when the needle portion 13H protruding from the second hole 113 toward the distal end side are pulled toward the proximal end side, the needle portion 13H is not caught in the edge portion of the distal end of the second hole 113. Therefore, a second advancing operation and a second retreating operation can be performed smoothly.

According to the disclosure, it is possible to provide an endoscope treatment tool, and a treatment method, which are capable of performing local injection satisfactorily while maintaining the performance of tissue incision and dissection.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An endoscope treatment tool, comprising:
a sheath including a first hole opened at a distal end of the sheath;
an electrode inserted into the first hole, the electrode including a second hole opened at a distal end of the electrode; and
a needle inserted into the second hole,
wherein the needle is movable to protrude from a distal end of the second hole of the electrode, and
wherein a gap between the electrode and the needle is configured to pass a fluid.

2. The endoscope treatment tool according to claim 1, wherein the needle includes:
a needle body with a sharp distal end, the needle body having a first outer diameter, and a flange having a second outer diameter that is larger than the first outer diameter,
wherein the endoscope treatment tool further comprises:
a tube having a distal end portion connected to the electrode, the tube translatable in the sheath in a first direction to advance toward a distal end of the sheath and a second direction toward a proximal end of the sheath, and
a transmission member located within the tube and connected to the flange, the transmission member translatable relative to the tube, and
wherein the tube includes an internal channel that forms a passage communicating with the gap.

3. The endoscope treatment tool according to claim 2, wherein the tube is a first tube, and the first tube comprises:
a second tube, and
a connecting portion,
wherein the connecting portion is fixed to an outer peripheral surface of the electrode, and a proximal end portion of the connecting portion is connected to a distal end portion of the second tube,
wherein the sheath includes:
a sheath body, and
a distal end part fixed to a distal end portion of the sheath body,
wherein the endoscope treatment tool further comprises:
a first slider connected to a proximal end portion of the first tube,
wherein the endoscope treatment tool has a first mode and a second mode, and
wherein, in the first mode, the first slider is translated in the first direction to a first state in which a distal end of the connecting portion abuts a proximal end of the distal end part and, in the second mode, the first slider is translated in the second direction to a second state in which a distal end portion of the electrode abuts a distal end of the distal end part.

4. The endoscope treatment tool according to claim 2, wherein the tube is a first tube, and the first tube comprises:
a connecting portion fixed to an outer peripheral surface of the electrode, and
a second tube connected to a proximal end portion of the connecting portion,
wherein the endoscope treatment tool further comprises:
a second slider connected to a proximal end portion of the transmission member,
wherein the endoscope treatment tool has a third mode and a fourth mode,
wherein, in the third mode, the second slider is translated in the first direction to a third state in which the needle protrudes from the distal end of the electrode and the flange abuts a proximal end of the electrode and,
wherein, in the fourth mode, the second slider is translated in the second direction to a fourth state in which the needle is housed on a proximal end side of the distal end of the electrode and the flange abuts a distal end of the second tube, and
wherein the passage is formed between the flange and the connecting portion.

5. The endoscope treatment tool according to claim 2, wherein the needle is a solid body.

6. The endoscope treatment tool according to claim 1, wherein the sheath includes:
a sheath body, and
a distal end part fixed to a distal end portion of the sheath body,
wherein the first hole is formed in the distal end part, and
wherein the endoscope treatment tool further comprises:
a tube having a distal end portion connected to the electrode, the tube translatable in the sheath body in a first direction to advance toward a distal end of the sheath and a second direction toward a proximal end of the sheath, and
a transmission member located within the tube and connected to the proximal end portion of the needle, the transmission member translatable relative to the tube.

7. The endoscope treatment tool according to claim 6, wherein a proximal end portion of the electrode is located inside the tube, and
wherein, in a retracted state, a distal end of the needle is housed inside the tube.

8. The endoscope treatment tool according to claim 1, wherein, in a retracted state, a distal end of the needle is housed inside the electrode and, in an extended state, the distal end of the needle protrudes from the distal end of the electrode.

9. The endoscope treatment tool according to claim 6, wherein a space between an inner peripheral surface of the sheath body and an outer peripheral surface of the tube is a passage through which the fluid flows, and
wherein the passage communicates with the inside of the tube.

10. The endoscope treatment tool according to claim 1, wherein the electrode is a knife configured to incise a tissue by being energized with a high frequency current.

11. The endoscope treatment tool according to claim 1, wherein at least one of an inner peripheral surface of the electrode and a surface of the distal end of the needle includes an insulating coating.

12. The endoscope treatment tool according to claim 1, wherein a material forming the needle is an insulating material.

13. The endoscope treatment tool according to claim 2, wherein the transmission member is a wire

14. A treatment method, comprising:
inserting an endoscope treatment tool according to claim 1 into a body;
with the needle in an extended state in which the needle protrudes from the distal end of the second hole of the electrode, making a cut from a mucosal surface to a submucosal layer by a cutting edge of the needle;
bulging the mucosal surface by injecting a fluid from an inside of the electrode to the submucosal layer via the cut; and
with the needle in a retracted state in which a distal end of the needle is housed on a proximal end side of a distal end of the electrode, dissecting the submucosal layer while energizing the electrode with a high frequency current.

15. The treatment method according to claim 14, wherein bulging the mucosal surface includes injecting the fluid from inside the electrode to the submucosal layer via the cut while the distal end of the electrode is pressed against the mucosal surface at the cut.

16. The treatment method according to claim 14, wherein bulging the mucosal surface includes injecting the fluid from inside the electrode to the submucosal layer via the cut while the distal end of the electrode is inserted into the cut.

17. The treatment method according to claim 14, further comprising:
with the needle in the retracted state, forming a marking sign on the mucosal surface by energizing the electrode with the high frequency current.
